(19) United States
(12) Patent Application Publication
(10) Pub. No.: US 2010/0121242 A1
(43) Pub. Date: May 13, 2010

(54) Knee protective assembly

(75) Inventor: Pang-Ching Chiang, Taipei (TW)

Correspondence Address: COOPER & DUNHAM, LLP, 30 Rockefeller Plaza, 20th Floor, NEW YORK, NY, 10112, US

(73) Assignee: PLUS MEDITECH CO., LTD.

(21) Appl. No.: 12/291,736

(22) Filed: Nov. 13, 2008

(51) Int. Cl. A61F 5/00

(52) U.S. Cl. 602/26

(57) ABSTRACT

A knee protective assembly has a mounting device and a protective device. The mounting device has two mounting frames and two jointing elements. Each mounting frame has a mounting panel, two articulated arms, a connecting tab and an adjusting mount with multiple adjusting holes. The jointing elements are respectively connected to the articulated arms of the mounting frames. The protective device is detachably connected to the mounting device and has two protective pads. The protective pads are respectively connected to the mounting frames of the mounting device and each protective pad has two inserting rods and two fasteners. Accordingly, after loosening the fasteners from the inserting rods on the upper protective pad and inserting the inserting rods into another adjacent adjusting holes of the upper mounting frame, the length of mounting device of the knee protective assembly is adjusted to fit with different users' legs.

### BACKGROUND OF THE INVENTION

1. Field of the Invention

The present invention relates to a protective assembly, and more particularly to a knee protective assembly that is mounted around the thigh and the shank of a user with an adjustable protection capability to keep the knee from being injured when the user acts with an unsuitable pose.

2. Description of Related Art

The ligament is clustering fibrous tissue and is used to connect the joints or the bones of the human body. When a user acts with an unsuitable pose, the ligament may be pull and drag overly and suddenly and this may cause the ligament strain or sprain. Conventional protective assembly is used to keep the ligament of a user from being injured and can be mounted around the knee, the wrist or the leg of the user. With reference to FIG. 7, a conventional knee protective assembly (30) is mounted around the thigh and the shank of a user and comprises a shank covering pad (32), a jointing element (33) and a thigh covering pad (31). The shank covering pad (32) is curved, is mounted around the shank of a user and has a top, a bottom, a side edge and two shank belts (35). The shank belts (35) are respectively connected to the top and the bottom of the shank covering pad (32) to hold the shank covering pad (32) on the shank of a user. The jointing element (33) is connected to the side edge of the shank covering pad (32) near the top and has a top end.

The thigh covering pad (3 1) is curved, is mounted around the thigh of a user and is connected to the jointing element (32) and has a top end, a bottom end, a side edge, an adjusting segment (34) and two thigh belts (36). The adjusting segment (34) is connected to the bottom end of the thigh covering pad (31), is connected to the side edge of the jointing element (33) near the top end to let the thigh covering pad (31) rotate relative to the shank covering pad (32). The thigh belts (36) are respectively connected to the top end and the bottom end of the thigh covering pad (31) to hold the thigh covering pad (31) on the thigh of a user.

However, the conventional knee protective assembly (30) always has fixed a length between the covering pads (31, 32) and cannot be adjusted in sizes to fit with the different people's legs, so the protective efficiency of the conventional knee protective assembly (30) is limited and cannot provide a preferred protection to the knee of a user.

To overcome the shortcomings, the present invention tends to provide a knee protective assembly to mitigate the aforementioned problems.

### SUMMARY OF THE INVENTION

The main objective of the present invention is to provide a knee protective assembly that is mounted around the thigh and the shank of a user with a preferred protection capability to keep the knee from being injured when the user acts with an unsuitable pose.

The knee protective assembly in accordance with the present invention has a mounting device and a protective device. The mounting device has two mounting frames and two jointing elements. Each mounting frame has a mounting panel, two articulated arms, a connecting tab and an adjusting mount with multiple adjusting holes. The jointing elements are respectively connected to the articulated arms of the mounting frames. The protective device is detachably connected to the mounting device and has two protective pads. The protective pads are respectively connected to the mounting frames of the mounting device and each protective pad has two inserting rods and two fasteners.

Other objectives, advantages and novel features of the invention will become more apparent from the following detailed description when taken in conjunction with the accompanying drawings.

### BRIEF DESCRIPTION OF THE DRAWINGS

FIG. 1 is a perspective front view of a knee protective assembly in accordance with the present invention for a person's leg;

FIG. 2 is an enlarged exploded perspective view of the knee protective assembly in FIG. 1;

FIG. 3 is another enlarged exploded perspective view of the knee protective assembly in FIG. 1;

FIG. 4 is a top view in partial section of the knee protective assembly in FIG. 1;

FIG. 5 is an operational rear perspective view of the knee protective assembly in FIG. 1;

FIG. 6 is an operational side view of the knee protective assembly in FIG. 1; and

FIG. 7 is a side view of a conventional knee protective assembly in accordance with the prior art.

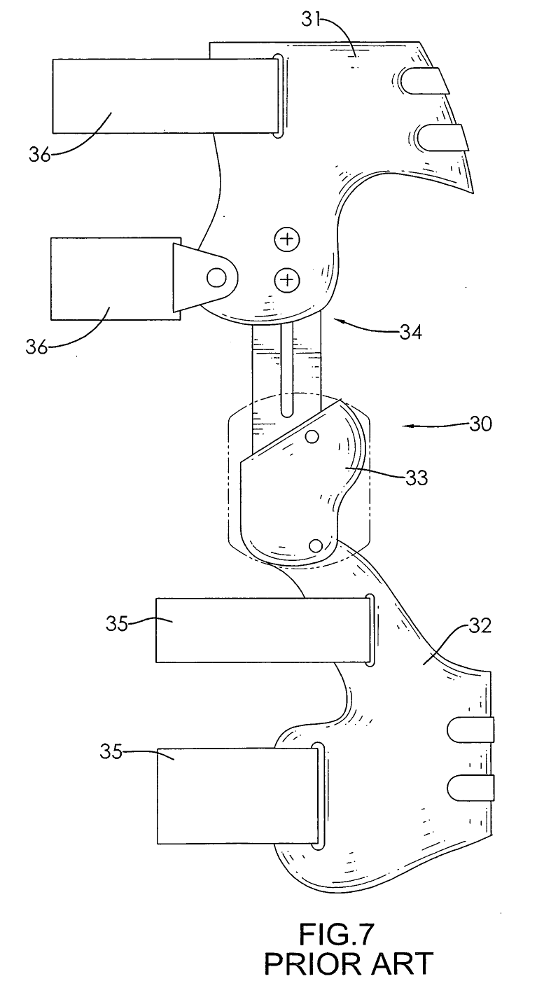

### DETAILED DESCRIPTION OF THE PREFERRED EMBODIMENT

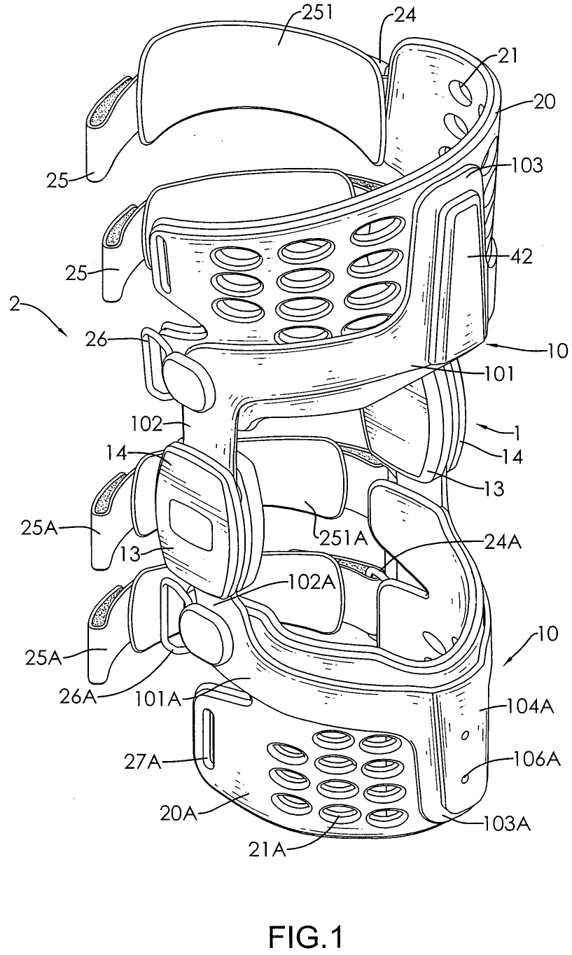

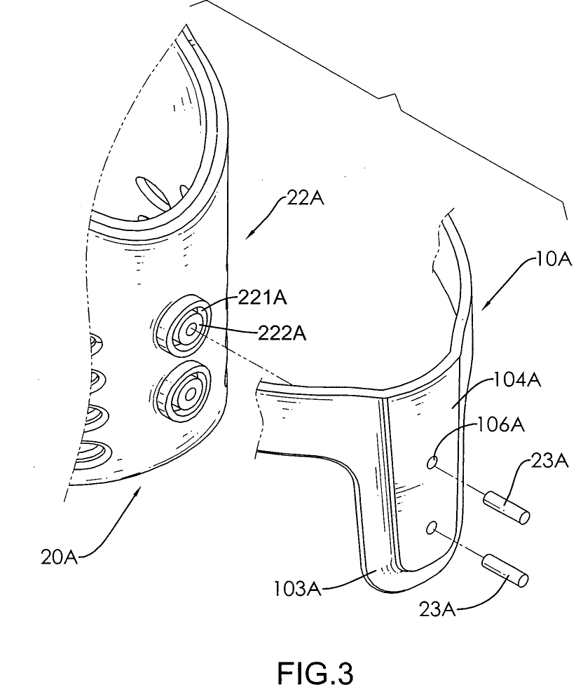

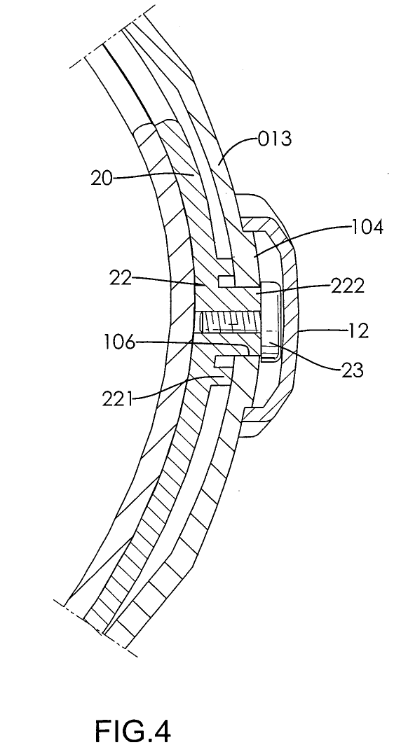

With reference to FIGS. 1 to 4, a knee protective assembly in accordance with the present invention can protect a person's knee and comprises a mounting device (1) and a protective device (2).

The mounting device (1) is used to mount around the thigh and the shank of a user and has an upper mounting frame (10), a cover (12), two jointing elements (14) and a lower mounting frame (10A).

The upper mounting frame (10) is mounted around the thigh of a user and has a mounting panel (101), two articulated arms (102), a connecting tab (103) and an adjusting mount (104).

The mounting panel (101) is curved, is mounted around the thigh of a user and has two free ends, a middle and an external surface.

The articulated arms (102) are respectively formed on and protrude downward from the free ends of the mounting panel (101) and are parallel to each other, and each articulated arm (102) has a lower end.

The connecting tab (103) is formed on and protrudes upward from the external surface of the mounting panel ( 101) near the middle.

The adjusting mount (104) is formed on the connecting tab (103) and has an elongated groove (105) and multiple adjusting holes (106). The elongated groove (105) is formed through the adjusting mount (104), the connecting board (103) and the external surface of the mounting panel (101). The adjusting holes (106) are formed through the adjusting mount (104), the connecting board (103) and the external surface of the mounting panel (101) at intervals and communicate with the elongated groove (105).

The cover (12) is detachably mounted around the adjusting mount (104) on the connecting board (103) to cover the elongated groove (105) and the adjusting holes (106).

The jointing elements (14) are respectively and pivotally connected to the articulated arms (102) of the upper mounting frame (10) and each jointing element (14) has a top end, a bottom end, an inner side and a flexible mat (13). The top ends of the jointing elements (14) are respectively connected to the lower ends of the articulated arms (102). The flexible mats (13) are respectively mounted on the inner sides of the jointing elements (14).

The lower mounting frame (10A) is mounted around the shank of a user, is pivotally connected to the jointing element (14) and has a mounting panel (101A), two articulated arms (102A), a connecting tab (103A) and an adjusting mount (104A).

The mounting panel (101A) is curved, is mounted around the shank of a user and has two free ends, a middle and an external surface.

The articulated arms (102A) are respectively formed on and protrude upward from the free ends of the mounting panel (101A) and are parallel to each other, and each articulated arm (102A) has an upper end. The upper ends of the articulated arms (102A) are respectively and pivotally connected to the bottom ends of the jointing elements (14).

The connecting tab (103A) is formed on and protrudes downward from the external surface of the mounting panel (10A) near the middle.

The adjusting mount (104) is formed on the connecting tab (103A) and has two adjusting holes (106A). The adjusting holes (106A) are formed through the adjusting mount (104A), the connecting tab (103A) and the external surface of the mounting panel (101A) at an interval.

The protective device (2) is detachably connected to the mounting device (1) and has an upper protective pad (20) and a lower protective pad (20A).

The upper protective pad (20) is curved, is detachably connected to the upper mounting frame (10) and has an external surface, two free ends, multiple through holes (21), a connecting segment (22), two fasteners (23), two holding rings (24, 26), two belt holes (27) and two thigh belts (25).

The through holes (21) are formed through the upper-protective pad (20).

The connecting segment (22) is formed on the external surface of the upper protective pad (20) and aligns with the adjusting mount (104) and has an external surface and two inserting rods (221). The inserting rods (221) are formed on and protrude from the external surface of the connecting segment (22) and are mounted in two adjusting holes (106) of the adjusting mount (104), and each inserting rod (221) has a threaded hole (222).

The fasteners (23) are respectively screwed into the threaded holes (222) in the inserting rods (221) to connect the upper protective pad (20) with the upper mounting frame (10).

The holding rings (24, 26) are respectively connected to the free ends of the upper protective pad (20).

The belt holes (27) are respectively formed through the free ends of the upper protective pad (20) and align with the holding rings (24, 26).

The thigh belts (25) are connected to the free ends of the upper protective pad (20) between the holding rings (24, 26) and the belt holes (27) and each thigh belt (25) has an inner side, a connecting end, a pasting end and a holding pad (251). The connecting end of one of the thigh belts (25) is connected to one of the holding rings (24, 26) and the connecting end of the other thigh belt (25) is connected to the belt hole (27) that aligns with the corresponding holding ring (24, 26). The pasting ends of the thigh belts (25) are connected to the other holding ring (26) and the other belt hole (27) away from the connecting ends of the thigh belts (25) and each pasting end of the thigh belt (25) may be a velcro. The holding pads (251) are respectively mounted on the inner sides of the thigh belts (25) between the connecting ends and the pasting ends.

The lower protective pad (20A) is curved, is detachably connected to the lower mounting frame (10A) and has an external surface, two free ends, multiple through holes (21A), a connecting segment (22A), two mounting rods (23A), two holding rings (24A, 26A), two belt holes (27A) and two shank belts (25A).

The through holes (21A) are formed through the lower protective pad (20A).

The connecting segment (22A) is formed on the external surface of the lower protective pad (20A) and aligns with the adjusting mount (104A) of the lower mounting frame (10A) and has an external surface and two inserting rods (221A). The inserting rods (221A) are formed on and protrude from the external surface of the connecting segment (22A) and are mounted in the adjusting holes (106A) of the adjusting mount (104A) of the lower mounting frame (10A) and each inserting rod (221A) has a mounting hole (222A).

The mounting rods (23A) are respectively screwed into the mounting holes (222A) in the inserting rods (221A) to connect the lower protective pad (20A) with the lower mounting frame (10A).

The holding rings (24A, 26A) are respectively connected to the free ends of the lower protective pad (20A).

The belt holes (27A) are respectively formed through the free ends of the lower protective pad (20A) and align with the holding rings (24A, 26A).

The shank belts (25A) are connected to the free ends of the lower protective pad (20A) between the holding rings (24A, 26A) and the belt holes (27A) and each shank belt (25A) has an inner side, a connecting end, a pasting end and a holding pad (251A). The connecting end of one of the shank belts (25A) is connected to one of the holding rings (24A, 26A) and the connecting end of the other shank belt (25A) is connected to the belt hole (27A) that aligns with the corresponding holding ring (24A, 26A). The pasting ends of the shank belts (25A) are connected to the other holding ring (26A) and the other belt hole (27A) away from the connecting ends of the shank belts (25A) and each pasting end of the shank belt (25A) may be a velcro. The holding pads (251A) are respectively mounted on the inner sides of the shank belts (25A) between the connecting ends and the pasting ends.

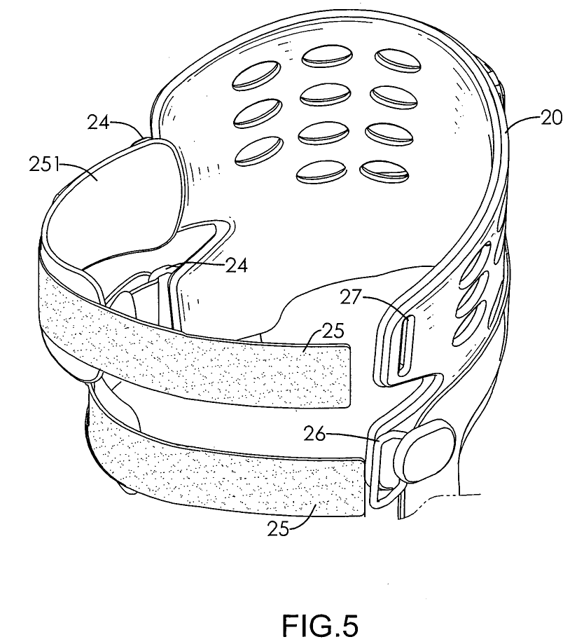

With reference to FIGS. 1 and 5, when the knee protective assembly is mounted on a leg of a user, the upper protective pad (20) is mounted on the thigh of the user, the lower protective pad (20A) is mounted on the shank of the user and the flexible mats (13) are mounted on the knee of the user to make the knee of the user be positioned between the mounting frames (10, 10A). Then, the belts (25, 25A) are connected to the corresponding belt holes (27, 27A) or the corresponding holding rings (26, 26A) to hold the knee protective assembly with the leg of the user near the knee to provide a protective efficiency.

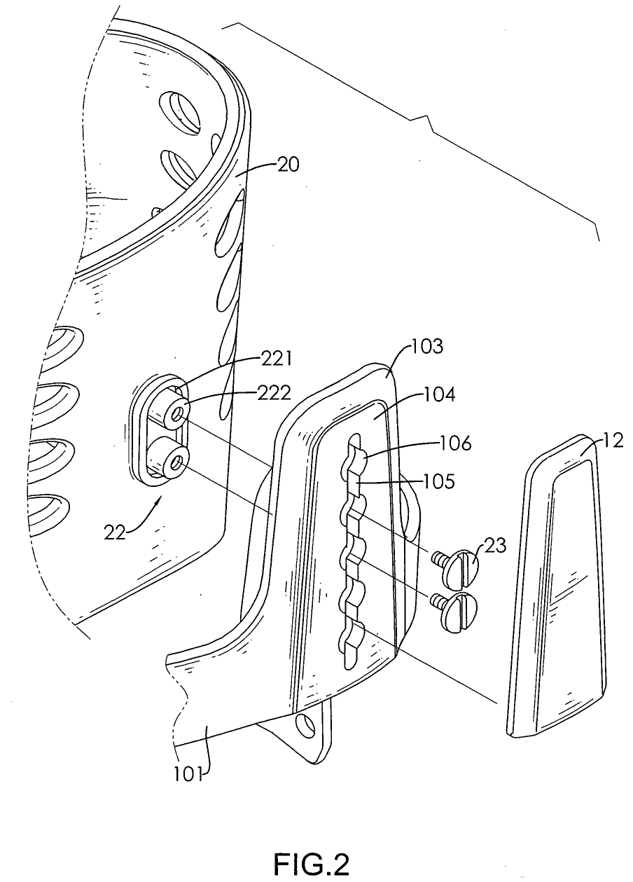

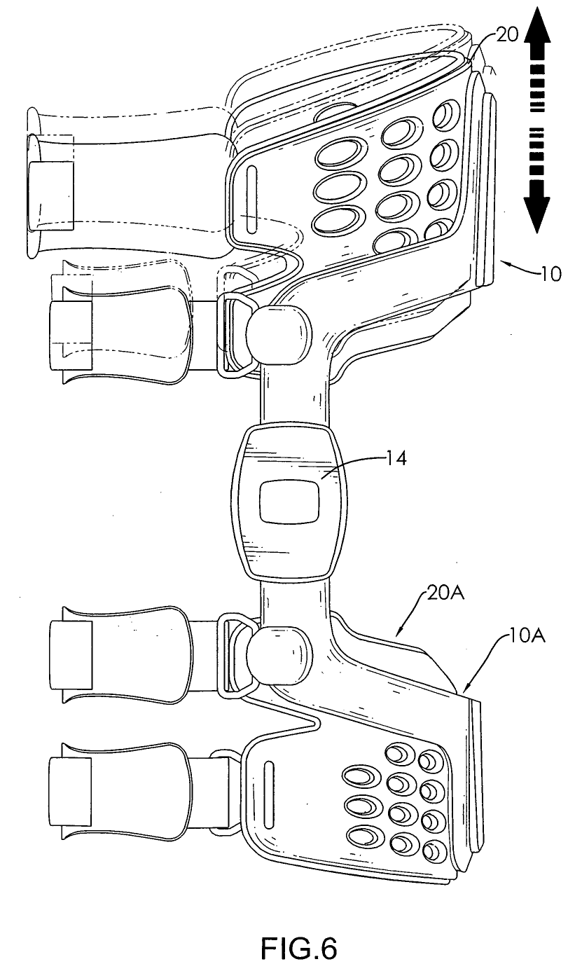

With further reference to FIGS. 2 and 6, when the knee protective assembly in accordance with the present invention is used, a user only needs to loosen the fasteners (23) from the threaded holes (222) of the inserting rods (221) on the upper protective pad (20) and insert the inserting rods (221) in another adjacent adjusting holes (106) of the upper mounting frame (10) to adjust the length of mounting device (1) of the knee protective assembly to fit with the different people's legs. Consequently, the present invention provides an easy way to adjust the length of the mounting device (1) of the knee protective assembly in sizes and a perfected protection capability.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. A knee protective assembly being curved and having
a mounting device being adapted to mount around a thigh and a shank of a user and having
an upper mounting frame being adapted to mount around the thigh of a user and having
a mounting panel being curved and having
two free ends;
a middle; and
an external surface;
two articulated arms being respectively formed on and protruding downward from the free ends of the mounting panel and parallel to each other, and each articulated arm having a lower end;
a connecting tab being formed on and protruding upward from the external surface of the mounting panel near the middle; and
an adjusting mount being formed on the connecting tab and having multiple adjusting holes formed through the adjusting mount, the connecting tab and the external surface of the mounting panel at intervals;
two jointing elements being respectively and pivotally connected to the articulated arms of the upper mounting frame and each jointing element having
a top end being connected to the lower end of a corresponding articulated arm;
a bottom end; and
an inner side;
a lower mounting frame being adapted to mount around the shank of a user, being pivotally connected to the jointing element and having
a mounting panel being curved and having
two free ends;
a middle; and
an external surface;
two articulated arms being respectively formed on and protruding upward from the free ends of the mounting panel and parallel to each other, and each articulated arm having an upper end pivotally connected to the bottom end of a corresponding jointing element;
a connecting tab being formed on and protruding downward from the external surface of the mounting panel near the middle; and
an adjusting mount being formed on the connecting tab and having two adjusting holes formed through the adjusting mount, the connecting tab and the external surface of the mounting panel at an interval; and
a protective device being detachably connected to the mounting device and having
an upper protective pad being curved, being detachably connected to the upper mounting frame and having
an external surface;
two free ends;
a connecting segment being formed on the external surface of the upper protective pad and aligning with the adjusting mount of the upper mounting frame and having
an external surface; and
two inserting rods being formed on and protruding from the external surface of the connecting segment and being mounted in two of the adjusting holes of the adjusting mount of the upper mounting frame, and each inserting rod having a threaded hole; and
two fasteners being respectively screwed into the threaded holes in the inserting rods to connect the upper protective pad with the upper mounting frame;
at least one thigh belt being connected to the free ends of the upper protective pad; and
a lower protective pad being curved, being detachably connected to the lower mounting frame and having
an external surface;
two free ends;
a connecting segment being formed on the external surface of the lower protective pad and aligning with the adjusting mount of the lower mounting frame and having
an external surface; and
two inserting rods being formed on and protrude from the external surface of the connecting segment and being mounted in the adjusting holes of the adjusting mount of the lower mounting frame, and each inserting rod having a mounting hole;
two mounting rods being respectively screwed into the mounting holes in the inserting rods to connect the lower protective pad with the lower mounting frame; and
at least one shank belt being connected to the free ends of the lower protective pad

2. The knee protective assembly as claimed in claim 1, wherein the adjusting mount of the upper mounting frame further has an elongated groove being formed through the adjusting mount, the connecting tab and the external surface of the mounting panel and communicating with the adjusting holes of the upper mounting frame.

3. The knee protective assembly as claimed in claim 2, wherein the mounting device further has a cover being detachably mounted on the adjusting mount on the connecting tab of the upper mounting frame to cover the elongated groove and the adjusting holes.

4. The knee protective assembly as claimed in claim 3, wherein each protective pad further has multiple through holes being formed through the protective pad.

5. The knee protective assembly as claimed in claim 4, wherein each jointing element further has a flexible mat mounted on the inner side of the jointing element.

6. The knee protective assembly as claimed in claim 5, wherein the upper protective pad has
two holding rings being respectively connected to the free ends of the upper protective pad;
two belt holes being respectively formed through the free ends of the upper protective pad and aligning with the holding rings; and
two thigh belts being connected to the free ends of the upper protective pad between the holding rings and the belt holes and each thigh belt having
an inner side;
a connecting end and the connecting end of one of the thigh belts being connected to one of the holding rings and the connecting end of the other thigh belt being connected to the belt hole that aligning with the corresponding holding ring;
a pasting end and the pasting ends of the thigh belts being connected to the other holding ring and the other belt hole away from the connecting ends of the thigh belts; and
a holding pad being mounted on the inner side of the thigh belt between the connecting end and the pasting end.

7. The knee protective assembly as claimed in claim 5, wherein the lower protective pad has
two holding rings being respectively connected to the free ends of the lower protective pad;
two belt holes being respectively formed through the free ends of the lower protective pad and aligning with the holding rings; and
two shank belts being connected to the free ends of the lower protective pad between the holding rings and the belt holes and each thigh belt having
an inner side;
a connecting end and the connecting end of one of the shank belts being connected to one of the holding rings and the connecting end of the other shank belt being connected to the belt hole that aligning with the corresponding holding ring;
a pasting end and the pasting ends of the shank belts being connected to the other holding ring and the other belt hole away from the connecting ends of the shank belts; and
a holding panel being mounted on the inner side of the thigh belt between the connecting end and the pasting end.
